(19) United States
(12) Patent Application Publication
(10) Pub. No.: US 2019/0056255 A1
(43) Pub. Date: Feb. 21, 2019

(54) MONITORING DEVICE FOR SUBJECT BEHAVIOR MONITORING

(71) Applicant: KONINKLIJKE PHILIPS N.V., EINDHOVEN (NL)

(72) Inventors: RONALDUS MARIA AARTS, GELDROP (NL); JACOBUS MARIA ANTONIUS VAN DEN EERENBEEMD, NEUNEN (NL)

(21) Appl. No.: 15/758,791

(22) PCT Filed: Sep. 14, 2016
(86) PCT No.: PCT/EP2016/071607

§ 371 Date: Mar. 9, 2018

(30) Foreign Application Priority Data
Sep. 18, 2015 (EP) 15185807.3

(51) Int. Cl. G01F 15/075; E03B 7/07; A61B 5/11; G01F 1/66

(52) U.S. Cl. CPC G01F 15/0755; E03B 7/07; E03B 7/04; G01F 1/666; A61B 5/1126

(57) ABSTRACT

A subject behavior monitoring device is for monitoring the behavior of a subject based on their usage of a plurality of fluid outlets of a fluid supply system in a building. An acoustic measurement unit is adapted to generate an acoustic monitoring signal which varies in dependence on a fluid flow from the plurality of fluid outlets. A signal processor processes the acoustic monitoring signal and is adapted to detect usage of each of the plurality of fluid outlets and determine which usages were carried out by the subject being monitored. In this way, the behavior of a subject, in terms of their usage of fluid outlets, such as water taps and gas appliances, can be detected and also discriminated from other occupiers of the building in which the subject is resident.

### FIELD OF THE INVENTION

The invention relates to a device for monitoring the behavior of one or more subjects, in particular their appliance usage habits. The subjects being monitored may be patients at home, or in a care home, or just elderly or frail people for who some monitoring is of value.

### BACKGROUND OF THE INVENTION

Subjects who are resident at home, in particular the elderly, need to be monitored for their wellbeing. It is well known to implement special measures in their house such as cameras, PIR-sensors, radar, etc. All these sensors enable a remote caregiver to have an impression on the behavior of the subject.

It has for example been recognized that one area which needs monitoring to determine the suitability of a subject to remain living at home is their personal hygiene activities in the bathroom. The article “Bathroom Activity Monitoring Based on Sound” by Jianfeng Chen et. al., Pervasive 2005, LNCS 3468. pp. 47-61, 2005 discloses the monitoring of sounds in the bathroom in order to distinguish between different activities, such as showering, flushing a toilet, washing hands and urinating.

It has also been proposed to monitor the water flow from taps and other water outlets in order to detect leaks or to track water usage. It is known that such monitoring may be based on the different sounds made when water flows from those outlets. This approach is for example disclosed in US 2013/0179095.

Approaches which aim to detect leaks or detect water consumption are not able to provide information which is mapped to a particular user, for example a subject who is being monitored.

### SUMMARY OF THE INVENTION

There is therefore a need for a device for monitoring a subject based on their usage pattern of a fluid from one or more fluid outlets, and which could enable differentiation between the subject and other users of the fluid outlets.

The aforementioned need is at least partly fulfilled with the invention. The invention is defined by the independent claims. The dependent claims define advantageous embodiments.

In the invention, a fluid can mean a condensed fluid or a gas. Preferably it is a condensed fluid as with these, the monitoring signal in e.g. an acoustic signal is of improved intensity or quality. The invention provides a sensor device/system, for instance a subject behavior monitoring device/system, which can detect a flow of a fluid from a fluid outlet. The system can distinguish between different points of usage in complex fluid networks with multiple branches and/or taps usage points. Such systems among others include household plumbing, buildings heating system, factory fuel system etc. In addition, the system is able to distinguish between different users. A user can be a person such as patient, caregiver, elder child etc. Typically it is an elder or patient in a home or hospital environment.

The fluid supply system is for example a water supply system. For example, a water system in a home can contain a shower, dishwasher, washing machine, kitchen sink, downstairs toilet and many more points of usage. The invention proposed provides a system that is able to recognize the individual fluid outlets in use at any moment in time. The system may be installed at a central point where the water supply pipe (or gas supply pipe) enters a building. A sensor records a flow signal from the household plumbing near the entrance into the home.

In an embodiment of the present invention, a device, for instance a subject behavior monitoring device, is provided for monitoring the behavior of a subject based on their usage of a plurality of fluid outlets of a fluid supply system in a building, or a vessel.

Suitable signal processing is used to give a fingerprint which can be used to determine the point of usage as well as to determine if the usage has been conducted by the subject being monitored. For example, the way a tap is opened reveals characteristics about the user, e.g. some people open a tap with less force than others. This type of difference may be used to identify users if there is more than one user (for example including a caregiver).

Furthermore, a change in the behavior of a user may be tracked over time, for example a progressive weakening, suggesting increasing frailty of the elderly.

The system can be adapted to have a database for storing historic or predetermined monitoring signals or parameters determinable from the monitoring signals by the processing of the signals characteristic for a usage event of one or more of the fluid outlets by one or more users. The actually generated monitoring signals or the determined parameters therefrom can be compared with these corresponding historic or predetermined data to determine which user is associated with a usage event.

The system does not need any external signal source as part of the system, and it can be implemented with a single or small number of detectors, which results in a reduced complexity and expense of the device.

The measurement unit can include a pressure sensor or accelerometer. It is for example a microphone adapted to generate the monitoring signal. It may do so in dependence of a pressure activity within a fluid supply pipe as caused by usage of any one or more of a fluid outlet.

The measurement unit may be provided at a single location at a supply pipe of the fluid supply system. This simplifies the installation of the system as well as reducing the cost of the system compared to a system with multiple sensors at the fluid outlets. The single location can be at a main supply pipe of the supply system.

The signal processor for example comprises a spectrum analysis system. This detects patterns in the frequency spectrum of the sound signal. The spectral analysis may for example make use of a Cepstrum filter.

The signal processor may comprise a neural network. This enables the system to evolve with use. A user interface is then provided, adapted to receive a user input for training the neural network.

Examples in accordance with a second aspect of the invention provide a subject behavior monitoring method for monitoring the behavior of a subject based on their usage of a plurality of fluid outlets of a fluid supply system in a building.

The monitoring signal may be generated in response to an acoustic signal. This signal may vary in dependence of a pressure activity within a fluid supply pipe. The monitoring signal may however be generated at a single location at a supply pipe of the fluid supply system.

The invention also provides a computer program which comprises computer program code means, adapted to perform the method defined above when said program is run on a computer. The invention can then be implemented in software to enable use of a known device such as e.g. a computer device equipped with a microphone to perform monitoring. Preferably the computer device is connected to a central computer via any kind of wired or wireless communication to transfer usage data to the central computer. The central computer can be remote, such as located at a location outside the building or vessel. This other location can be that of a caregiver or central operating or steering premises.

The computer program may thus for example run on a Personal Computer, laptop, tablet, mobile phone etc. A microphone as the measurement unit installed nearby of a supply network and is in communication with the computer.

### BRIEF DESCRIPTION OF THE DRAWINGS

An example of the invention will now be described in detail with reference to the accompanying drawings, in which:

FIG. 1 shows a subject behavior monitoring device;

FIG. 2 shows a sound signal showing a tap opening and closing event;

FIG. 3 shows a neural network for data analysis; and

FIG. 4 shows a flowchart illustrating a method for monitoring a movement of a fluid.

### DETAILED DESCRIPTION OF EMBODIMENTS

The invention provides a subject behavior monitoring device for monitoring the behavior of a subject based on their usage of a plurality of fluid outlets of a fluid supply system in a building. A measurement unit is adapted to generate a monitoring signal which varies in dependence on a fluid flow from the plurality of fluid outlets. A signal processor processes the monitoring signal and is adapted to detect usage of each of the plurality of fluid outlets and determine which usages were carried out by the subject being monitored. In this way, the behavior of a subject, in terms of their usage of fluid outlets, such as water taps and gas appliances, can be detected and also discriminated from other occupiers of the building in which the subject is resident.

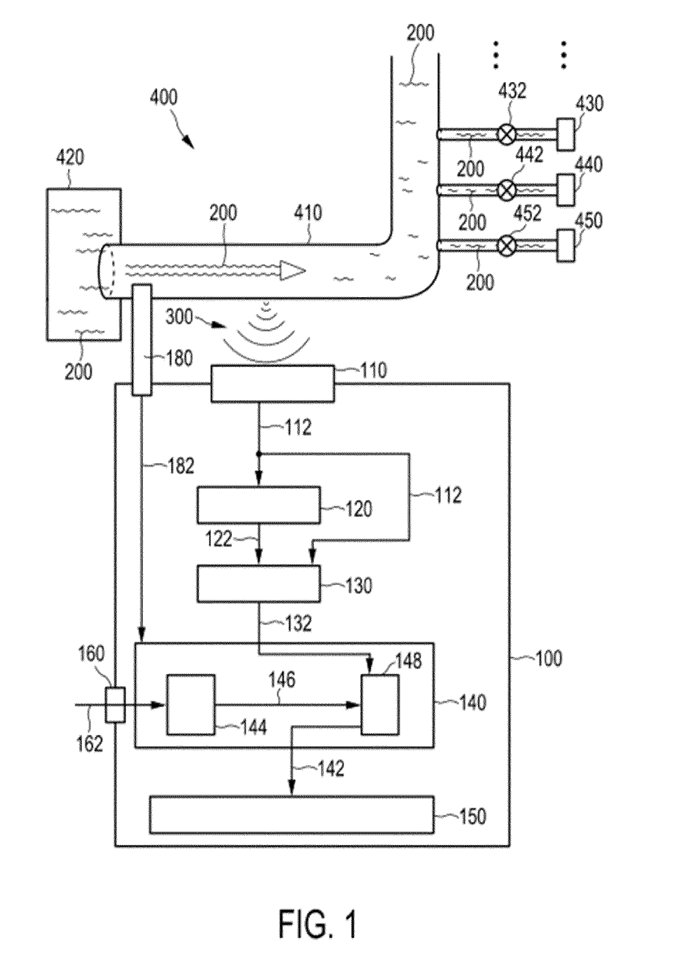

FIG. 1 shows schematically a representation of the monitoring device 100, for instance a subject behavior monitoring device, for monitoring a movement of a fluid 200.

The main example involves monitoring a water system, and the fluid is then water. The device is for example for use in monitoring the water flow from taps or other outlets (toilets, showers etc.) within the plumbing of a building. However, the fluid 200 may for be gas, oil or other fuel.

The fluid 200 is guided in the supply network 400, which for a water system comprises the plumbing of a building, in the form of pipes 410. A plurality of consumption units 430, 440, 450 is connected to the supply network 400 by means of respective taps 432, 442, 452. The taps are linked to the pipe 410 via a supply node 420. The supply network 400 can for instance be found in a household, such as a private household, an office building or a facility building. It shall be understood that the supply network 400 for guiding the fluid 200 is not part of the monitoring device 100.

Due to a movement of the fluid 200, a flow signal 300 originates from the supply network 400. The flow signal 300 originating from the supply network 400 is in most cases a physical pressure or stress, for instance appearing as a travelling wave, such as sound or vibration. That travelling wave may originate either from the fluid 200 itself or from a housing part of the supply network 400 and may also be caused, for instance, by mechanical movements of parts of the supply network 400, such as opening or closing a valve of a fluid consumption unit connected to the supply network 400.

In many situations, a consumption unit connected to the supply network via a tap regulates its fluid consumption by means of operating a valve. The flow signal 300 can also be dependent on a velocity or a flux of the fluid 200 in the supply network 400. The flow signal 300 originating from the supply network 400 is dependent of a movement of the fluid 200 that is guided by the supply network 400. A certain movement of the fluid 200 results in a change of the flow signal 300, wherein such change is characteristic for the certain movement. For example, if the fluid 200 moves, a pressure wave originates due to a friction between the fluid 200 and the supply network 400. That pressure wave significantly depends on the manner of the movement.

A measurement unit 110 of the monitoring device 100 is preferentially positioned in close distance to at least a part of the supply network 400. Preferentially, the measurement unit 110 of the monitoring device 100 is in physical contact with the supply network 400. The measurement unit 110 can be any sensor being adapted to record the flow signal 300 and to generate a monitoring signal 112. It may be an acoustic sensor such as a microphone, or a vibration sensor such as an accelerometer. Preferentially, the measurement unit 110 or the device comprises an analog-to-digital converter for converting the analog flow signal 300 into a digital monitoring signal 112.

In a preferred embodiment of the monitoring device 100, for instance a subject behavior monitoring device, the measurement unit 110 is a microphone adapted to generate the monitoring signal 112 in dependence on a pressure activity originating from the supply network 400. If a consumption unit 430, 440, 450 connected to the supply network 400 via a tap 432, 442, 452 causes a movement of the fluid 200, that movement of fluid 200 usually effects a travelling pressure or stress wave originating from the supply network 400. Preferentially, the measurement unit 110 is therefore a microphone. It shall be understood that microphone can be any kind of sensor that measures a pressure or stress wave, such as sound or vibration. The generated monitoring signal 112 is preferentially an audio signal. This has the advantage that surveillance of an entire pipe network, for instance to be found in a household, can be performed by listening to noises emerging from a single point in the supply network typically near a supply node 420.

In another example of the monitoring device 100, the measurement unit 110 is a sensor adapted to generate the monitoring signal 112 in dependence of a velocity of the fluid 200. A consumption unit 430, 440, 450 connected to the supply network 400 via a tap 432, 442, 452 causes a movement of the fluid 200 which results in a change of the velocity of the fluid 200. The time dependent rate of change of velocity of the fluid 200 can also be indicative for a movement caused by a certain consumption unit and is therefore an appropriate flow signal 300 to monitor.

In one embodiment, the generated monitoring signal 112 is forwarded directly to a determination unit 130. In another embodiment, the monitoring device 100 comprises a memory unit 120 for storing the monitoring signal 112. In this embodiment, the determination unit 130 receives a stored monitoring signal 122. This has the advantage that the measurement unit 110 and determination unit 130 can operate in a time-decoupled manner.

The determination unit 130 of the monitoring device 100 is adapted to analyze the generated monitoring signal 112 or the stored monitoring signal 122 by determining a value of a predefined parameter of the generated monitoring signal 112, wherein the determined value of the predefined parameter is indicative for a shape of the generated monitoring signal 112. Such a predefined parameter can for instance be a base frequency of the monitoring signal 112, an average amplitude of the monitoring signal 112, a total harmonic distortion value of the monitoring signal 112, a standard deviation to the average amplitude, a power spectral density in a certain frequency range of the monitoring signal 112, a rate of change of the monitoring signal 112, a Cepstrum Coefficient, a Mel Filter Cepstrum Coefficient.

A respective parameter is thus at least partially indicative of a shape of the monitoring signal 112. Preferentially, the determination unit is adapted to determine a value of each of a plurality of predefined parameters.

The determination unit 130 forwards the determined value/values 132 to the mapping unit 140 of the monitoring device 100. The mapping unit 140 is adapted to map the determined value/values 132 to one of a plurality of events, wherein a respective one of the plurality of events corresponds to a characteristic movement of the fluid 200. Such an event can, for instance, be caused by a certain consumption unit 430, 440, 450 that is connected to the supply network 400 via a tap 432, 442, 452. The monitoring device 100 is thus adapted to distinguish between different points of usage in a complex fluid supply network 400, for example to be found in a household.

Since therefore a certain movement in the fluid 200 is mapped to a certain event that has caused the movement in the fluid 200, the monitoring device 100 is adapted to provide information about a consumption behavior. This information is used to monitor the behavior of a subject, in particular who is at risk of losing the ability to conduct simple household functions. In particular, the information is used to ensure that a subject is maintaining an ability to conduct personal hygiene tasks.

An advantage of the monitoring device 100 is its low complexity set up: Only one measurement unit 110 is needed for surveillance of an entire supply network 400 comprising a plurality of taps 432, 442, 452. Also, no additional sources are needed.

A further advantage of the monitoring device 100 is its simple installation: The measurement unit 110 of the monitoring device 100 does not have to be installed inside the supply network 400, but is for example positioned on an outer surface or near to an outer surface of the supply network 400.

The monitoring device 100 is very well suited for monitoring the movement of water and/or gas at supply node 420 of a household, such as a private household, an office building, a facility building. The monitoring device 100 preferentially comprises a display 150 for displaying mapping results 142 to the user and thus to assist a user or their caregiver in increasing an awareness of the consumption behavior.

For mapping the determined values 132 to one of a plurality of events, the mapping unit 140 preferentially employs a neural network (not shown in FIG. 1). Preferentially, the monitoring device 100 comprises a user interface 160 adapted to receive a user input 162 for training the neural network. Therefore, a user (or the system installer/administrator) can program the neural network and adapt the monitoring device 100 to his demands.

In another embodiment, the mapping unit 140 additionally comprises a storing unit 144 for storing a list comprising a plurality of entries, wherein a respective entry of the list is a predefined value or a predefined combination 146 of values of the one or more predefined parameters associated with one of the plurality of events. In this embodiment, the mapping unit 140 further comprises a comparison unit 148 for comparing the determined value/values 132 with the predefined value or the predefined combination 146 of values of the list, wherein the mapping unit 140 is adapted to map the determined value/values 132 to one of the plurality of events in dependence of a result of the comparison.

In one embodiment, the comparison unit 148 is adapted to carry out the comparison by means of predefined criteria of similarity. Preferentially, the mapping unit 140 is adapted to map the determined value/values 132 to that of the plurality of events, where the result of the comparison shows a highest degree of similarity between the determined value/values 132 and the predefined value or the predefined combination of values associated with the event. In this embodiment, the user interface 160 preferentially serves for a user programming of the list of the storing unit 144. This allows a user to adapt the monitoring device 100 to his demands.

The above described embodiment of the mapping unit 140 where the mapping unit 140 employs a comparison unit 148 for mapping the determined values 132 to one of a plurality of events is well suited, if the generated monitoring signal 112 monitors the velocity and/or flux of the fluid 200 or, respectively, a signal substantially proportional to the velocity or flux of the fluid 200.

The monitoring device 100 optionally comprises a flow-rate sensor 180 adapted to generate a flow-rate signal 182 in dependence of a flux of the fluid 200. In this embodiment, in addition to a mapping of a fluid 200 movement to a consumption unit, a total fluid consumption is determined. This allows generating an overview of an overall consumption behavior. The flow-rate sensor 180 therefore forwards the flow-rate signal 182 to the mapping unit 140. Advantageously, the flow-rate signal 182 is supplied to the display 150 together with the mapping results 142.

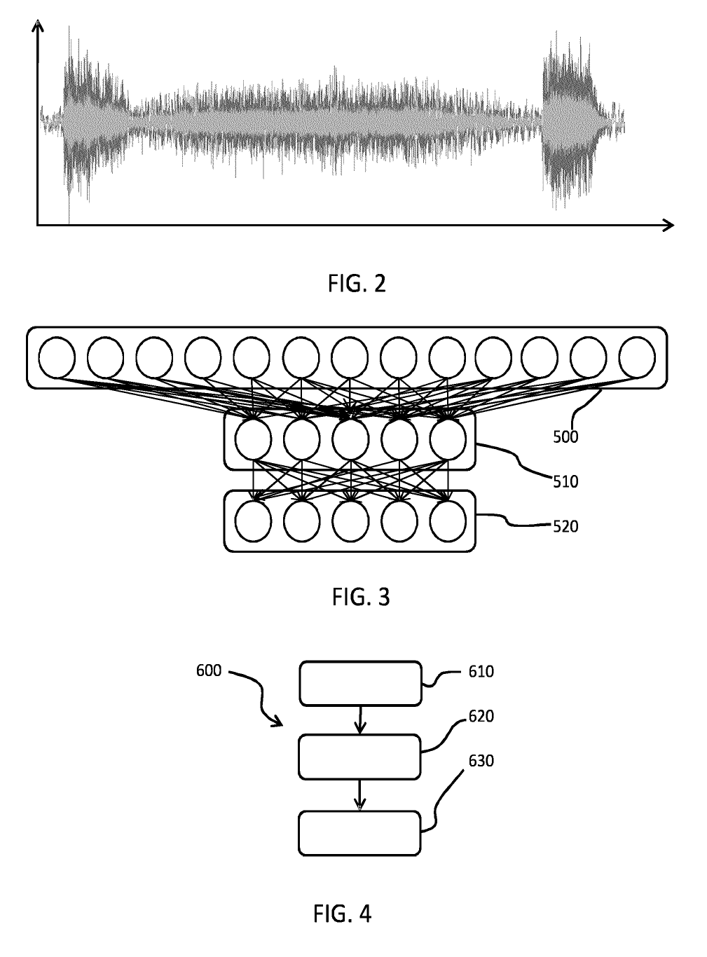

FIG. 2 shows an example of a microphone signal mounted at a water pipe adjacent the water meter, while a tap somewhere in the house in opened. The plot show a tap opening event, a flow event and a tap closing event at sequential points in time.

The signal is decomposed into different spectral features using band pass signal processing as part of a spectral analysis of the signal.

The feature extraction is for example based on the use of Mel Filter Cepstral Coefficients. This is a representation of the short-term power spectrum of a sound, based on a linear cosine transform of a log power spectrum on a nonlinear Mel scale of frequency.

A cepstrum is the result of taking the Inverse Fourier Transform (IFT) of the logarithm of the estimated spectrum of a signal. The Mel-frequency cepstral coefficients (MFCCs) are coefficients that collectively make up a Mel Filter Cepstrum. The difference between the standard Cepstrum and the Mel-Frequency Cepstrum is that in the MFC, the frequency bands are equally spaced on the Mel scale, which approximates the human auditory system's response more closely than the linearly-spaced frequency bands used in the normal cepstrum. This frequency warping can allow for better representation of sound, for example, in audio compression and it provides a compact representation of the spectrum.

The MFCCs are for example derived by:

taking the Fourier transform of a windowed excerpt of a signal;

mapping the powers of the spectrum obtained onto the Mel scale, using triangular overlapping windows;

taking the logs of the powers at each of the Mel frequencies;

taking the discrete cosine transform of the list of Mel log powers, as if it were a signal.

The MFCCs are the amplitudes of the resulting spectrum.

The system may simply derive the cepstrum. For this purpose, the determination unit divides the monitoring signal 112 into a plurality of samples. For one sample, the determination unit calculates the power spectrum as the square of its Fast Fourier Transform with a fixed number of sample points. The determination unit further links the calculated spectrum to a fixed number of power coefficients using a filter bank. Each power coefficient is the logarithm of the power transferred through one filter. Finally, the determination unit determines the Cepstrum Coefficients by forming the discrete cosine transform of the power coefficients.

In order to determine Mel Filter Cepstrum Coefficients (MFCCs) as mentioned above, the frequencies of the filters used for the calculation of the MFCCs are additionally arranged to be evenly spaced on the scale of pitches as perceived by human beings.

An event detected can for instance be a consumption process caused by a consumption unit such as a washing machine, a shower, a toilet, a basin, a heater. Such a respective consumption process causes a unique movement of the fluid which is characteristic for the consumption process. Due to the movement of the fluid, a sound, in most cases a pressure wave, originates from the supply network. The measurement unit is adapted to generate the monitoring signal in dependence of the sound. Therefore, the shape of the monitoring signal is indicative of the movement of the fluid and therefore indicative for an event that has caused the movement. As mentioned above, the shape of the monitoring signal can be described by one or more predefined parameters, such as a base frequency of the monitoring signal, an average amplitude of the monitoring signal, a total harmonic distortion value of the monitoring signal, a standard deviation to the average amplitude, a Cepstrum Coefficient.

Some events will give the same flow information same regardless of the user that has initiated the event, for example operating a washing machine. However, even these events may give information about the user, based on the time of day at which the event was performed or the duration of the event. However, others events will give information about the characteristics of the user based on the signal analysis of the event (regardless of the time at which it takes place). The device of the invention provides analysis of at least one event which can be attributed to a particular user based on the characteristics of the monitored signal.

Such events for example include turning on a tap (basin, shower or bath), or turning off the tap. The time between turning on and turning off may also give information about the user.

Other events may also be detected based on the sound or vibrations transmitted to the water or gas pipe system, and which may also be of interest for analysis. For example, walking on floors and stairs may give characteristic profiles (particularly for wooden or other suspended floors), and when these are followed by other events (like taking a shower or flushing a toilet) it becomes possible to infer who has just taken the shower or flushed the toilet even if this user information could not be detected in isolation.

A gas appliance with a user-controlled knob (rather than an on-off button) will also give user specific information. Thus, the invention is not limited to a water system but is also of interest for a gas system. Note that gas usage patterns can also be detected based on sounds in the water pipes as they are for example interconnected by a gas heating system.

The storing unit is adapted to store a plurality of predefined values of the predefined parameter or, respectively, a plurality of predefined combinations of values of a plurality of predefined parameters, each associated with one of the plurality of events and also associated with the particular user to be identified when the nature of the signal itself conveys information about the user.

In one example implementation of the system, 13 MFCCs are obtained (corresponding to 13 features) taken from 15 ms sample windows of sound data, with windows sampled every 5 ms.

As mentioned above, the system may make use of a neural network to implement a learning process to enable extracted features to be recognized.

FIG. 3 shows 13 inputs 500 to a set of 5 neural network neurons 510. Thus, data sets are taken from 5 taps for training a Feed Forward Neural Network with the 13 inputs. The neurons (which are a hidden layer with the neural network) feed 5 outputs filters 520.

The neural network enables the way a tap is opened to reveal information about the user, and this helps identifying persons if there is more than one user (including the caregiver). The use of a neural network is particularly effective if the determination unit determines a Cepstrum Coefficient (or Mel Cepstrum Coefficient) of the generated monitoring signal.

Cepstrum Coefficients form a n elements vector \vec{p} that is the input to the neural network. As shown in FIG. 3, the neural network comprises a number of hidden layers, shown together as 510, and an output layer 520. Each layer can be represented by a matrix operation. A first layer with a first output vector \vec{a}₁ is described by equation (1)

\vec{a₁}=f₁(W_{1,1}·\vec{p}+\vec{b₁}) (1)

The input vector \vec{p} is multiplied by a m x n weight matrix W _{1,1} and a m elements first bias vector \vec{b}₁ is added. A first transfer function f₁ works on each element of the resulting vector and determines the first output vector \vec{a}₁ of the first layer. A next layer with a second output vector \vec{a}₂ takes \vec{a}₁ as an input and is described by equation (2)

\vec{a₂}=f₂(W_{2,1}·\vec{a₁}+\vec{b₂}) (2)

where f₂ is a second transfer function and \vec{b}₂ a second bias vector. The following layers are determined in that iterative mode. The last layer forms the output layer.

The dimension of the output vector of the output layer is equal to the number of the plurality of events that the mapping unit is adapted to distinguish. For suitable weight matrices and bias vectors, the output vector of the output layer predicts the event that caused the input vector \vec{p}.

In a preferred embodiment as shown in FIG. 3, the neural network comprises two layers, one hidden layer and an output layer. Preferentially, the hidden layer employs a hyperbolic tangent function as the first transfer function f₁ and the output layer a linear function as the second transfer function f₂ .

Preferentially, the mapping unit 140 is adapted to apply a training algorithm to the neural network for improving the mapping. The training algorithm is applied to set the elements of the weight matrices and bias vectors. The training algorithm processes a list comprising a plurality of entries, wherein a respective entry of the list is a predefined value of the predefined parameter or a predefined combination of values of the predefined parameters that is determined from a monitoring signal associated with a known event.

For more detailed description of programming a neural network, reference is made to the following publication: Cernazanu et. al., “Training Neural Networks Using Input Data Characteristics”, Advances In Electrical And Computer Engineering vol. 8 (2) 2008 p: 65-70.

As mentioned briefly above, the monitoring device has a user interface top enable training of the neural network. A user can in this way program the neural network and adapt the monitoring device.

Training the neural network can for instance work as follows:

A user initiates an event, for instance by activating a certain consumption unit connected to the supply network and informs the monitoring device via the user interface, which event has occurred. The event causes a certain shape of the monitoring signal to be present.

The same event may be conducted by multiple different users who are resident in the same building, or are likely to be present in the building at times (such as a caregiver). In this way, the training not only enables identification of different events but also different users who are initiating those events.

The determination unit determines the value of the parameter or parameters derived from the monitoring signal. In this way, the neural network learns which determined value of the predefined parameter belongs to which event. The information obtained is stored in the storing unit 144.

If the monitoring device is installed for monitoring a different supply network or if a set-up of the supply network changes, for instance due to new or alternative consumption units, the list in the storing unit and/or the neural network of the mapping unit is preferentially re-trained via user inputs such that it is adapted to the new conditions of the different or, respectively, modified supply network.

As mentioned above, the monitoring device may additionally comprise a flow-rate sensor, adapted to generate a flow-rate signal in dependence of a flux of the fluid. In addition to a mapping of a fluid movement to an event, a total fluid consumption can then be determined. This allows an overview of consumption behavior. A total fluid consumption may instead be calculated from a typical consumption value that is associated with the identified events that have caused a fluid movement. For some events, the consumption value could vary, which could negatively influence the accuracy. However, accuracy is maintained if different consumption values of the same event cause differences in characteristics of the fluid movement that are distinguished by the mapping unit. Therefore, the monitoring device may be adapted to determine fluid consumption by summing up pre-known consumption values, wherein a respective consumption value is associated with a certain value of the predefined parameter.

FIG. 4 shows a flowchart illustrating an embodiment of a method 600 for monitoring a movement of a fluid in accordance with the second aspect of the invention.

In a first step 610, a monitoring signal is generated in dependence on a flow, for example a sound, wherein the sound originates from a fluid supply network. In many situations, the supply network is a pipe or a distribution point of a pipe system. Usually, a plurality of consumption units is connected to the supply network via taps. Due to the movement of the fluid caused by a consumption unit, such as a washing machine, a gas heating system, an oil consumer, a sound originates from the supply network. The sound may be a pressure wave or a change in velocity of the fluid. For example, this method step 610 can thus be carried by installing a measurement unit, such as a microphone or flux sensor, nearby the supply network and by recording the sound by means of the measurement unit.

In a second step 620, the monitoring signal is processed to detect usage events of each of the plurality of fluid outlets. These usage events are detected based on the shape of the generated monitoring signal. This method step 620 can be carried out by means a commonly known signal processing means, in particular with audio signal processing means. The shape of the generated monitoring may for example be determined based on a Cepstral Coefficient. The determined value describes a characteristic shape of the monitoring signal. In other words: In this way, a footprint in the generated monitoring signal is recognized.

In a third step 630, the determined signal is mapped to one of a plurality of events and users. Each event thus is associated with a characteristic fluid movement that directly causes a characteristic sound, such as a characteristic sound or a characteristic rate of change of the velocity of the fluid. Furthermore, for at least some events, initiation of the event by different users gives rise to different characteristics. The characteristic sound is expressed in the generated monitoring signal and captured by carrying out method step 820. By carrying out method step 830, the cause of the characteristic movement is determined. For carrying out the mapping, for instance a neural network can be employed.

Alternatively, the determined value is compared with a predefined value of the predefined parameter by means of criteria of similarity.

Implementation of the method 600 thus provides a user with information about consumption behavior, with at least some information specific to a particular user.

In the above described embodiments, certain equations are used for determining an output vector of the neural network that is employed by the mapping unit. The mapping unit is adapted to map a certain movement of the fluid to an event in dependence of the output vector. In other embodiments, other equations can be used for determining the output vector.

In the description above, water and gas are given as examples of fluids that may be monitored by the monitoring device. In other applications, different fluids, such as oil or fuel, are monitored.

The system may combine monitoring of multiple types of fluid (e.g. gas and water). The device then has a plurality of measurement units, for measuring a flow signal in two different fluid supply networks. In the situation of a household with a water supply and a gas supply, a first measurement unit generates a first monitoring signal in dependence of a sound of a water supply network and a second measurement unit generates a second monitoring signal in dependence of a sound of a gas supply network. This has the advantage that since both the movement of the first and the second fluid are monitored, the monitoring device is, for instance, adapted to distinguish whether gas is used for heating rooms or for heating water. The measurement unit is thus adapted to generate a monitoring signal in dependence of a sound originating from an overall supply network that supplies two or more fluids. In many situations, a water guidance system and a gas guidance system are mechanically coupled, for instance through connections onto a central heating unit. Vibrations generated by moving gas can thus penetrate the water and the housing of water pipes. Therefore, the monitoring signal expresses both the movement of the water and the gas. As explained above, the information of the movement of a plurality of fluids are advantageously combined in order to improve the mapping.

The monitoring device may be installed inside the supply network or in close distance to the supply network and may provide the monitoring signal by means of a cable or a wireless connection to the determination unit. The determination unit and the mapping unit may be part of a personal computer and may be installed in the same integrated circuit or in two separated integrated circuits. Furthermore, the function of the determination unit and/or the function of the mapping unit may be carried out by software running on a computer and employing processing units.

As explained above, the system may be implemented with a single sensor arrangement at a fluid supply pipe. Additional sensors may optionally also be deployed, for example hot water sensors at hot water outlets so that the usage of hot water may be tracked.

The device may be used in a private dwelling or in a care home or in any other residence where a person being monitored resides either permanently or temporarily.

A computer program which implements the signal analysis may be stored/distributed on a suitable medium, such as an optical storage medium or a solid-state medium supplied together with or as part of other hardware, but may also be distributed in other forms, such as via the Internet or other wired or wireless telecommunication systems.

Other variations to the disclosed embodiments can be understood and effected by those skilled in the art in practicing the claimed invention, from a study of the drawings, the disclosure, and the appended claims. A single unit or device may fulfill the functions of several items recited in the claims. The mere fact that certain measures are recited in mutually different dependent claims does not indicate that a combination of these measures cannot be used to advantage. Any reference signs in the claims should not be construed as limiting the scope. In the claims, the word “comprising” does not exclude other elements or steps, and the indefinite article “a” or “an” does not exclude a plurality.

1. A device for monitoring the behavior of a subject based on the subject's usage of one or more fluid outlets of a fluid supply system in a building or vessel, the device comprising:
a measurement unit for generating a monitoring signal which is dependent on a fluid flow in the fluid supply system caused by the usage of the one or more fluid outlets;
a signal processor for:
receiving the monitoring signal;
detecting usage events of the one or more fluid outlets from a shape of the monitoring signal; and
for at least one usage event comprising turning on or off a tap, attributing that usage event to the subject being monitored based on one or more characteristics of the shape of the monitoring signal.

2. The device of claim 1, wherein the measurement unit comprises or consists of a microphone for generating the monitoring signal.

3. The device of claim 1, wherein the measurement unit is for providing at a single location within the fluid supply system.

4. The device of claim 1, the supply system comprising at least one supply pipe and wherein the measurement unit is for attaching to at least one supply pipe of the supply system.

5. The device of claim 1, wherein the signal processor comprises a spectrum analysis system.

6. The device as claimed in of claim 5, wherein the signal processor is adapted to extract features using a Cepstrum filter.

7. The device of claim 5, wherein the signal processor comprises a neural network.

8. (canceled)

9. The device of claim 1 comprising the fluid supply system.

10. The device of claim 1 wherein the fluid supply system is any one of:
a water supply system
a chemical supply system
a fuel supply system
a heating system.

11. A method for monitoring behavior of a subject based on the subject's usage of one or more fluid outlets of a fluid supply system in a building or vessel, the method comprising the steps of:
generating a monitoring signal based on a fluid flow caused by the usage of the one or more fluid outlets;
processing the monitoring signal to:
detect usage events of the one or more fluid outlets based on a shape of the monitoring signal; and
for at least one usage event comprising turning on or off a tap, attributing that usage event to the subject being monitored-based on one or more characteristics of the monitoring signal.

12. The method of claim 11, comprising generating the monitoring signal in response to an acoustic signal.

13. The method of claim 11, comprising generating the monitoring signal at a single location within the fluid supply system.

14. The method of claim 11, wherein the processing comprises spectrum analysis using a Cepstrum filter.

15. The method of claim 14, wherein processing comprises using a neural network.

16. (canceled)

17. A computer program product comprising computer program code storable on, or stored on a computer readable medium, or downloadable from a communications network, which code, when run on a computer, causes the signal processor to perform the steps of any one of the methods as claimed in claim 11.
